(19) United States
(12) Patent Application Publication
(10) Pub. No.: US 2025/0241558 A1
(43) Pub. Date: Jul. 31, 2025

(54) ON-SENSOR COMBINATION OF DECISION TREE AND FINITE STATE MACHINE FOR FALL DETECTION

(71) Applicant: STMicroelectronics International N.V., Geneva (CH)

(72) Inventors: Swapnil Sayan SAHA, Mountain View, CA (US); Krishna Chaitanya PALLE HAYAGREEVA, Cupertino, CA (US); Derek LILIENTHAL, Marina, CA (US); Mahesh CHOWDHARY, San Jose, CA (US)

(73) Assignee: STMicroelectronics International N.V., Geneva (CH)

(21) Appl. No.: 18/423,902

(22) Filed: Jan. 26, 2024

(51) Int. Cl. A61B 5/11; A61B 5/00; G01P 15/08; G16H 40/63

(52) U.S. Cl. CPC A61B 5/1117; A61B 5/7267; G01P 15/08; G16H 40/63; A61B 2562/0219

(57) ABSTRACT

A method of detecting falls of human beings includes loading a decision tree on a sensor including an accelerometer, wherein the decision tree is trained based on automatic feature and filter selection and a size constraint for fitting into the sensor, to detect falls. The method also includes loading a finite state machine on the sensor, wherein the finite state machine is configured at least partly to identify phases of a fall independent from the decision tree. The method further includes inputting data derived from the accelerometer into the decision tree and the finite machine, and combining output from the decision tree and output from the finite state machine via the sensor to determine whether a fall has occurred.

### BACKGROUND

### Technical Field

The present disclosure relates to the field of human fall detection. The present disclosure relates more particularly to real-time fall detection based on a combination of decision tree and finite state machine, capable of being implemented on one or more sensors.

### Description of the Related Art

Falls are a leading cause of injury and death among older adults. According to the World Health Organization, approximately 28-35% of people aged 65 and over fall each year, increasing to 32-42% for those over 70 years of age. Falls can result in serious injuries, such as hip fractures, head injuries, and other fractures. Efficient and accurate fall detection remains a technological challenge.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which, in and of itself, may also be inventive.

### BRIEF SUMMARY

There exist a huge variety of types of falls, and each type has its own unique characteristics. This makes it difficult to develop a fall detection system that can accurately detect all types of falls. Additionally, false positives of detected falls are a major challenge that needs to be addressed in order to make fall detection systems more useable. Further, real-time fall detection systems need to be of small footprint, low-power and low cost to be accessible and utilitarian to all, as well as working for all normal activities of daily living.

The technology disclosed herein relates to human fall detection based on machine learning, e.g., primarily using on-sensor computation of decision tree and finite state machine (FSM) on one or more sensors. The size of the machine learning implementation in accordance with the technology disclosed herein can be sufficiently small to be partially or completely executed on wearable devices with small battery, consuming much less computational resource or power when compared with a fall detection system implemented on personal computers, general purpose processors, or the like.

Conventional fall detection technologies may have to use a combination of different sensors (e.g., accelerometer, gyroscope, pressure sensor, camera, or the like), which increases cost and power consumption, and add calibration and time synchronization burden. Conventional fall detection technologies tend to use complex algorithms that have a high requirement for computational resources, yet they may still be limited to detecting only certain type of falls (e.g., hard falls).

In comparison, embodiments of the presently disclosed technology are highly power efficient. For example, some implementations can be completely integrated into an ultra-low power smart accelerometer (e.g., generating only raw accelerometer readings for use), with a consumption of 50-170 uA. Therefore, embodiments of the presently disclosed technology can remain constantly running on a sensor for real-time fall detection, without necessarily waking up a general-purpose application processor (e.g., microcontroller), resulting in significantly extended battery life. Additionally, conventional machine learning algorithms can suffer from a high rate of false positives corresponding to non-fall events of vigorous nature such as jogging, jumping, dropping on a sofa, or the like. The presently disclosed technology can use one or more FSMs to strengthen the inference given by a machine learning algorithm (e.g., decision tree). The presently disclosed technology can detect different types of falls (e.g., falling in different directions, falling from chair, falling from standing, or the like) with high accuracy.

Embodiments of the present disclosure provide a computer-implemented method for fall detection, which includes: loading a decision tree on at least one sensor including an accelerometer, wherein the decision tree is trained based, at least in part, on automatic feature and filter selection and a size constraint for fitting within the target sensor memory and compute constraints, to detect falls of human beings; loading a finite state machine on the at least one sensor, wherein the finite state machine is configured, at least in part, to identify a set of phases of a fall independent from the decision tree; inputting data derived from at least the accelerometer into the decision tree and the finite machine; and combining output from the decision tree and output from the finite state machine via the at least one sensor to determine whether a fall has occurred.

Embodiments of the present disclosure provide a non-transitory computer-readable medium storing contents that cause one or more processors to perform actions. The actions include: implementing a decision tree that is trained based, at least in part, on automatic filter and feature selection over first motion data, to detect falls of human beings; implementing a finite state machine configured, at least in part, to identify a set of phases of a fall independent from the decision tree; inputting second motion data into the decision tree and the finite machine; and combining output from the decision tree and output from the finite state machine to determine whether a fall has occurred.

Embodiments of the present disclosure provide a system that includes one or more processors and a computing device coupled to the one or more processors and configured to perform actions. The actions include: implementing a decision tree that is trained based, at least in part, on automatic feature and filter selection over first motion data, to detect falls of human beings; implementing a finite state machine configured, at least in part, to identify a set of phases of a fall independent from the decision tree; inputting second motion data into the decision tree and the finite machine; and combining output from the decision tree and output from the finite state machine to determine whether a fall has occurred.

### BRIEF DESCRIPTION OF THE SEVERAL VIEWS OF THE DRAWINGS

FIG. 1A is a block diagram of a fall detection system, according to some embodiments.

FIG. 1B is a flow diagram illustrating a process for fall detection, according to some embodiments.

FIG. 2 illustrates an example process of the training and implementation of decision tree, accordingly to some embodiments.

FIG. 3 shows an example of the phases that an FSM is configured to identify, according to some embodiments.

FIG. 4 is a block diagram illustrating elements of an example computing device or system utilized in accordance with some embodiments of the techniques described herein.

### DETAILED DESCRIPTION

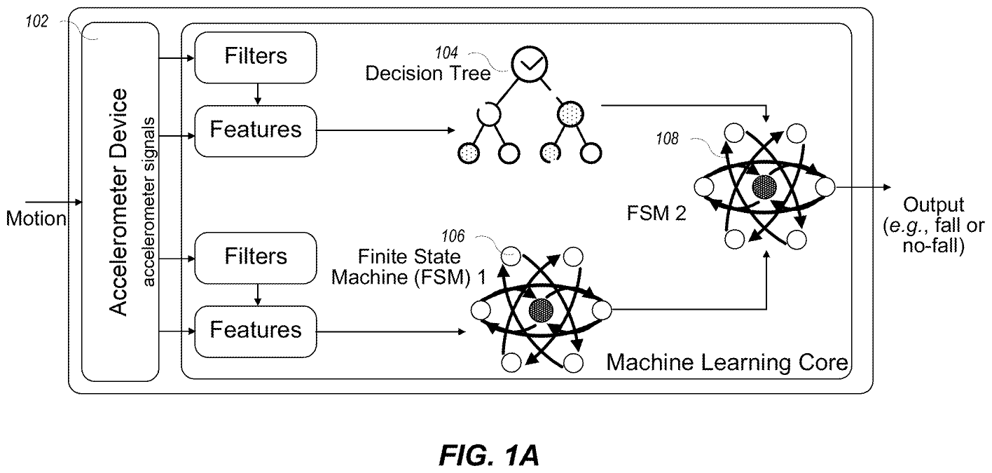

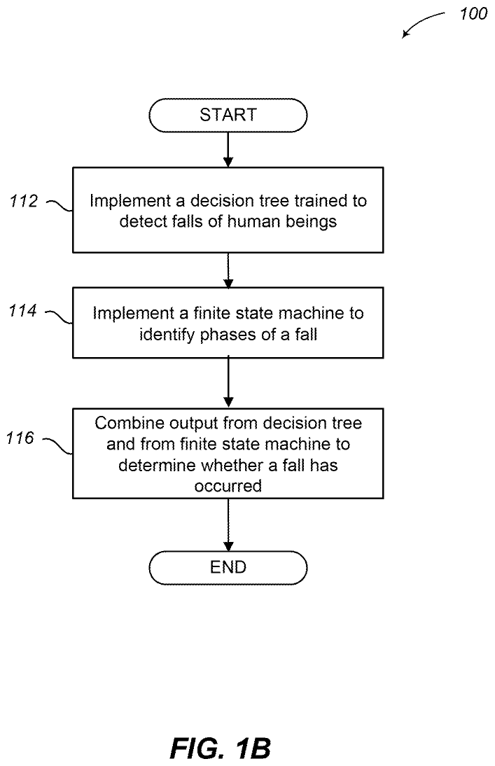

FIG. 1A is a block diagram of the fall detection system, according to some embodiments. The system includes an accelerometer device 102 (e.g., including one or more accelerometer dies) that generates accelerometer readings, measurements, or signals in accordance with motion sensed. Alternatively or in addition, the motion can be sensed by one or more other types of motion sensing devices (e.g., gyroscopes or magnetometers) that generate motion data different than accelerometer readings (e.g., gyroscope or magnetometer readings).

The motion data (e.g., accelerometer readings) are communicated to a decision tree 104 and a first FSM 106 via respective filters and feature selection mechanisms, using applicable communication connections (not shown). The communication connections may include one or more hardwires, one or more computer networks, one or more wired or wireless networks, satellite transmission media, one or more cellular networks, or some combination thereof. The communication connections may include a publicly accessible network of linked networks, possibly operated by various distinct parties, such as the Internet. The communication connections may include other network types, such as one or more private networks (e.g., corporate or university networks that are wholly or partially inaccessible to non-privileged users), and may include combinations thereof, such that (for example) one or more of the private networks have access to and/or from one or more of the public networks. Furthermore, the communication connections may include various types of wired and/or wireless networks in various situations, including satellite transmission. In addition, the communication connections may include one or more communication interfaces including radio frequency (RF) transceivers, cellular communication interfaces and antennas, USB interfaces, ports and connections (e.g., USB Type-A, USB Type-B, USB Type-C(or USB-C), USB mini A, USB mini B, USB micro A, USB micro C), other RF transceivers (e.g., infrared transceivers, connection interfaces based on IEEE 802.15.4 OpenThread protocol, Zigbee® protocol, or IEEE 802.15.4 MAC layer, Z-Wave® connection interfaces, wireless Ethernet (“Wi-Fi”) interfaces, short range wireless (e.g., Bluetooth®, Bluetooth® Low Energy (BLE)) interfaces or the like.

The output from the decision tree 104 and the output from the FSM 106 are combined to generate a final output of the fall detection system, indicating whether a fall has occurred or not. The combining can be implemented by a second FSM 108, by the first FSM 106 itself, by a non-FSM machine learning model (e.g., artificial neural network), or by other non-FSM computational methods. In some embodiments, the combination of the decision tree and FSM(s) is completely implemented within a sensor including the accelerometer device 102 (e.g., implemented within a machine learning core (MLC) of the sensor). In some embodiments, the combination of the decision tree and FSM(s) can be cloud-based or otherwise implemented in a software and/or hardware form on one or more computing devices including a microcontroller unit (MCU), computer, mobile device, tablet computer, smart phone, handheld computer, and/or workstation, etc.

FIG. 1B is a flow diagram illustrating a process 100 for fall detection, according to some embodiments. The process 100 can be implemented, in part or in whole, via a system such as the system shown in FIG. 1A. In some embodiments, at least part of the process 100 is performed in real-time relative to the motion sensed by or other interactions with the accelerometer device 102 (or other motion sensing device(s)).

At block 102, the process 100 includes implementing a decision tree, which is trained to detect falls of human beings. In some embodiments, the decision tree is trained based, at least in part, on automatic feature and filter selection and a size constraint for fitting into the at least one sensor (e.g., an accelerometer), and the decision tree is loaded on the sensor (e.g., in its machine learning core (MLC)).

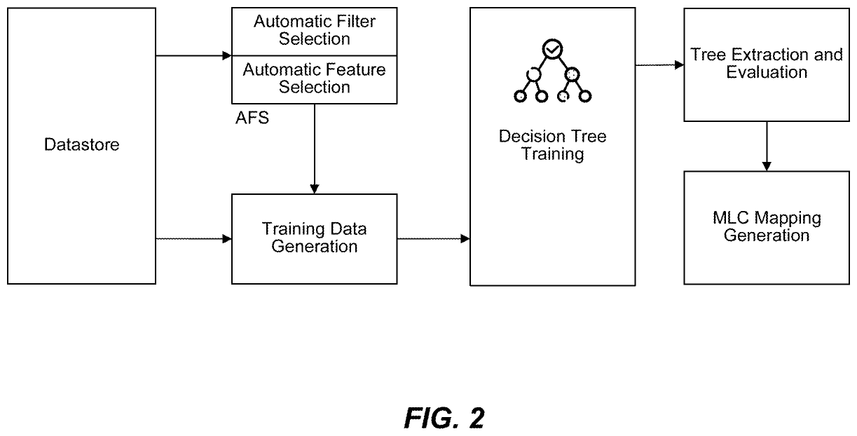

FIG. 2 illustrates an example process of the training and implementation of the decision tree. As shown in FIG. 2, a datastore contains base motion data for training the decision tree. As an example, to eliminate the need for model personalization and encompass a wide variety of daily activities, sensor position, sensor orientation, sensor model, terrain, geographic location, time-of-day, and person-to-person physiological variations, the base motion data can include labeled data from multiple public activities of daily living (ADL) datasets. The datastore contains traces of readings from accelerometers placed in many different locations on the human body with unrestricted sensor position and orientation. The base data can contain months of fall and no-fall ADL traces from thousands of participants (e.g., accelerometer readings resampled at 50 Hz and scaled at [−4g, +4g]). There are hundreds of different ADL mapped to various classes, collected at various times of the day, terrain, locations, and sensors.

The base motion data can be processed through applicable automatic filter selection and automatic feature selection, to generate training data for training the decision tree. Filter selection can be an entropy-based method that searches all combination of frequency bands (e.g., on logarithmic scale) and selects the frequency of interest (FOI) which minimizes the entropy against the other classes. Illustratively, an exhaustive search can be performed on accelerometer norm and multiple feature selection algorithms (e.g., random forest, AdaBoost, ANOVA, sequential feature selection, and recursive feature elimination) can be implemented.

A feature and filter file can be generated in accordance with the automatic filter selection and automatic feature selection. Illustratively, the feature and filter file can be a JavaScript Object Notation (JSON) file, including a list of optimal features and filters, as well as their parameters, shown below:
{
   “filters”:
      {
         “filter_name”: “filter_1”,
         “type”: “IIR2”,
         “input”: “Acc_V”,
         “f_low”: 0.4,
         “f_high”: 0.5,
         “b1”: 0.00624403504634285,
         “b2”: 0.0,
         “b3”:−0.00624403504634285,
         “a2”:−1.9843741615265953,
         “a3”: 0.9875119299073144,
         “gain”: 1.0
      }
   },
   “features”: {
      {
         “feaure_name”: “MEAN”,
         “input”: “Acc_V”
      },
      (
         “feature_name”: “ENERGY”,
         “input”: “Acc_V”
      {
         “feature_name”: “MINIMUM”,
         “input”: “Acc_V”
      }
      {
         “feature_name”: “ENERGY”,
         “input”: “Acc_Z”,
         “sign”: “signed”
      },
      {
         “feature_name”: “ENERGY”,
         “input”: “Acc_Y”,
         “sign”: “signed”
      },
      {
         “feature_name”: “MAXIMUM”,
         “input”: “Acc_V”
      },
      {
         “feature_name”: “ZERO_CROSSING”,
         “input”: “Acc_Y”,
         “threshold”: 0.00571735620432331,
         “sign”: “signed”
      },
      {
         “feature_name”: “MEAN”,
         “input”: “Acc_Y”,
         “sign”: “signed”
      },
      {
         “feature_name”: “MAXIMUM”,
         “input”: “Acc_Y”,
         “sign”: “signed”
      }

The content of the feature and filter file (e.g., the optimal feature and filter list) can be applied to the base motion data to generate training data for the decision tree. Illustratively, the generated training data can be one or more files in Attribute-Relation File Format (ARFF), including features extracted or derived (e.g., windowed raw datalogs) from the base motion data based on the automatic filter selection and automatic feature selection, which can be used during training and evaluation of the decision tree.

In various embodiments, the decision tree is trained with a size constraint for fitting into a sensor or system (e.g., to be executed in its entirety within the MLC thereof), such as the system shown in FIG. 1A. For example, the decision tree can be trained with its number of nodes limited to a threshold (e.g., 100, 200, or the like) and/or a maximum depth limited to a threshold (e.g., 50, 100, or the like), depending on sensor model or MLC capacity thereof. The trained decision tree can then be extracted and evaluated, e.g., against testing data. If the trained decision tree passes evaluation, the tree is implemented. In some embodiments, the implementation includes loading the tree on the MLC of a sensor or system, e.g., as shown in FIG. 1A. Illustratively, the decision tree, features, and filters can be mapped to appropriate registers within the MLC using an applicable configuration generator such as a Unico configuration file (UCF) generator, thereby loading the decision tree on the sensor.

Referring back to FIG. 1B, at block 114, the process 100 includes implementing an FSM to identify phases of a fall. In some embodiments, the FSM is loaded on the same sensor or system as the decision tree, whereas the FSM is configured, at least in part, to identify a set of phases of a fall independent from the decision tree. While the decision tree may detect the presence of falls very well, the decision tree-based detection can be falsely triggered by non-falls. To improve false positive rate, the FSM can provide a symbolic inference alongside the decision tree output; in other words, the FSM can be used to reinforce the detection of falls while rejecting false positives.

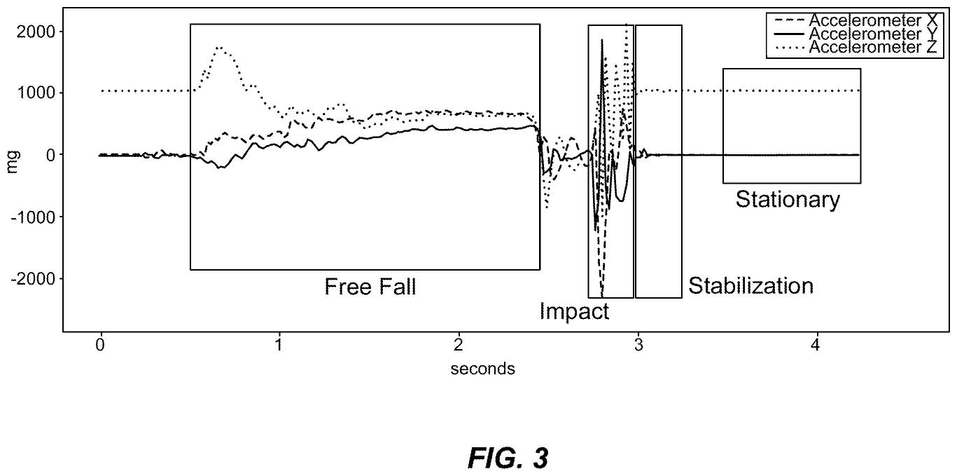

FIG. 3 shows an example of the phases (e.g., free fall, impact, stabilization, and stationary) the FSM can be configured to identify, e.g., based on motion data such as traces of accelerometer readings. The FSM can use various configurable parameters to enable fine grain control of its performance. In some embodiments, the hyperparameters for the FSM can be extrapolated or otherwise derived based on the base motion data as described with reference to FIG. 2. Similar to implementing the decision tree, in various embodiments, the FSM has a size constraint for fitting into a sensor or system (to be executed in its entirety within the MLC thereof), such as the system shown in FIG. 1A.

Referring back to FIG. 1B, at block 116, the process 100 includes receiving motion data (e.g., accelerometer readings) as input to the decision tree and to the FSM, and combining output from the decision tree and from the FSM to determine whether a fall has occurred and generate an output of fall detection in accordance with the determination. In some embodiments, the output from the decision tree and the output from the FSM are combined using another FSM, which may or may not reside in the same sensor or system as the decision tree or the FSM. In some embodiments, the output from the FSM is based, at least in part, on the output of the decision tree, whereby the FSM combines both outputs as an output of its own.

In some embodiments, the combining includes determining whether the finite state machine identifies the set of phases of a fall within a threshold of time of a fall being detected by the decision tree. For example, if all the phases (or other features) of a fall are detected by the FSM within a threshold period of time (e.g., 3 seconds) of a fall being detected by the decision tree, then the inference is classified as a fall; otherwise, it's a non-fall.

As described above, in some embodiments, the entirety or part of process 100 can be implemented within a sensor or system (e.g., an ultra-low power smart accelerometer consuming less than 0.3 mW of power (50-170 μA of current)) that senses motion. The technology disclosed herein can detect different kinds of falls (e.g., falling in different directions (left, right, backward, forward, lateral), falling with fall prevention attempt, falling from chair (in different directions), falling from standing, or the like) and can reject different kinds of false positives (e.g., jumping, throwing a ball in sports, pretending to fall on a soft sofa, or the like).

Those skilled in the art will appreciate that the various operations depicted via FIG. 1B, 2, 3, as well as those described elsewhere herein, may be altered in a variety of ways. For example, the particular order of the operations may be rearranged; some operations may be performed in parallel; shown operations may be omitted, or other operations may be included; a shown operation may be divided into one or more component operations, or multiple shown operations may be combined into a single operation, etc.

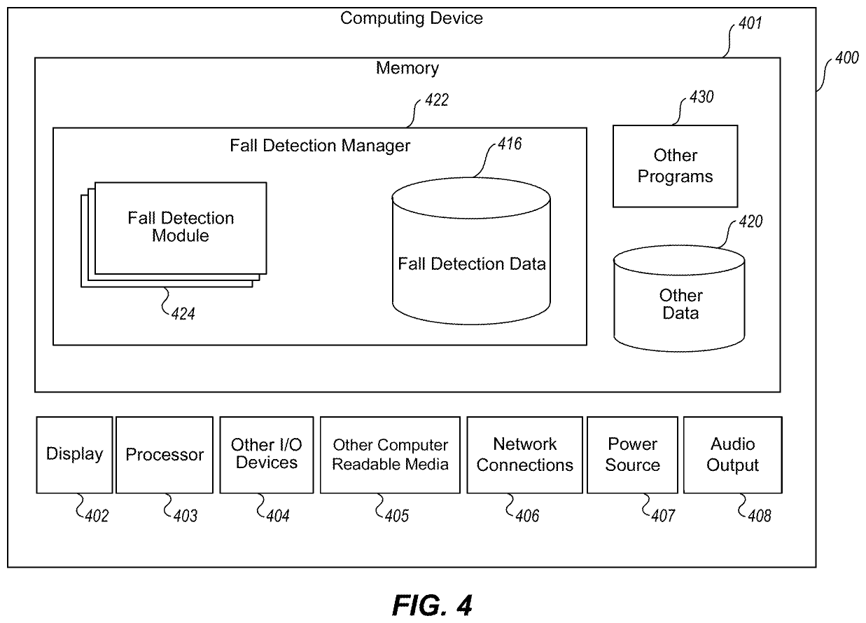

FIG. 4 is a block diagram illustrating elements of an example computing device or system 400 utilized in accordance with some embodiments of the techniques described herein. Illustratively, the computing device 400 corresponds to a computing device implementing the system of FIG. 1A, the process 100 of FIG. 1B, or at least a part thereof.

In some embodiments, one or more general purpose or special purpose computing systems or devices may be used to implement the computing device 400. In addition, in some embodiments, the computing device 400 may comprise one or more distinct computing systems or devices, and may span distributed locations. Furthermore, each block shown in FIG. 4 may represent one or more such blocks as appropriate to a specific embodiment or may be combined with other blocks. Also, the fall detection manager 422 may be implemented in software, hardware, firmware, or in some combination to achieve the capabilities described herein.

As shown, the computing device 400 comprises a computer memory (“memory”) 401, a display 402 (including, but not limited to a light emitting diode (LED) panel, cathode ray tube (CRT) display, liquid crystal display (LCD), touch screen display, projector, etc.), one or more Central Processing Units (CPU) or other processors 403, Input/Output (I/O) devices 404 (e.g., keyboard, mouse, RF or infrared receiver, universal serial bus (USB) ports, High-Definition Multimedia Interface (HDMI) ports, other communication ports, and the like), other computer-readable media 405, network connections 406, a power source (or interface to a power source) 407, and audio output 408 (including, but not limited to speakers, buzzers, handphones, etc.). The fall detection manager 422 is shown residing in memory 401. In other embodiments, some portion of the contents and some, or all, of the components of the fall detection manager 422 may be stored on and/or transmitted over the other computer-readable media 405. The components of the computing device 400 and fall detection manager 422 can execute on one or more processors 403 and implement applicable functions described herein. In some embodiments, the fall detection manager 422 may operate as, be part of, or work in conjunction and/or cooperation with other software applications stored in memory 401 or on various other computing devices. In some embodiments, the fall detection manager 422 also facilitates communication with peripheral devices via the I/O devices 404, or with another device or system via the network connections 406.

The one or more fall detection modules 424 is configured to perform actions related, directly or indirectly, to the photon data obtaining, model training, model deployment, surface fall detection, or other functions described herein. In some embodiments, the fall detection module(s) 424 stores, retrieves, or otherwise accesses at least some fall detection-related data on some portion of the fall detection data storage 416 or other data storage internal or external to the computing device 400. In various embodiments, at least some of the fall detection modules 424 may be implemented in software or hardware.

Other code or programs 430 (e.g., further data processing modules, communication modules, a Web server, and the like), and potentially other data repositories, such as data repository 420 for storing other data, may also reside in the memory 401, and can execute on one or more processors 403. Of note, one or more of the components in FIG. 4 may or may not be present in any specific implementation. For example, some embodiments may not provide other computer readable media 405, a display 402, or audio output 408.

In some embodiments, the computing device 400 and fall detection manager 422 include API(s) that provides programmatic access to add, remove, or change one or more functions of the computing device 400. In some embodiments, components/modules of the computing device 400 and fall detection manager 422 are implemented using standard programming techniques. For example, the fall detection manager 422 may be implemented as an executable running on the processor(s) 403, along with one or more static or dynamic libraries. In other embodiments, the computing device 400 and fall detection manager 422 may be implemented as instructions processed by a virtual machine that executes as one of the other programs 430. In general, a range of programming languages known in the art may be employed for implementing such example embodiments, including representative implementations of various programming language paradigms, including but not limited to, object-oriented (e.g., Java, C++, C#, Visual Basic.NET, Smalltalk, and the like), functional (e.g., ML, Lisp, Scheme, and the like), procedural (e.g., C, Pascal, Ada, Modula, and the like), scripting (e.g., Perl, Ruby, Python, JavaScript, VBScript, and the like), or declarative (e.g., SQL, Prolog, and the like).

In a software or firmware implementation, instructions stored in a memory configure, when executed, one or more processors of the computing device 400 to perform the functions of the fall detection manager 422. In some embodiments, instructions cause the one or more processors 403 or some other processor(s), such as an I/O controller/processor, to perform at least some functions described herein.

The embodiments described above may also use well-known or other synchronous or asynchronous client-server computing techniques. However, the various components may be implemented using more monolithic programming techniques as well, for example, as an executable running on a single CPU computer system, or alternatively decomposed using a variety of structuring techniques known in the art, including but not limited to, multiprogramming, multithreading, client-server, or peer-to-peer, running on one or more computer systems each having one or more CPUs or other processors. Some embodiments may execute concurrently and asynchronously, and communicate using message passing techniques. Equivalent synchronous embodiments are also supported by a fall detection manager 422 implementation. Also, other functions could be implemented and/or performed by each component/module, and in different orders, and by different components/modules, yet still achieve the functions of the computing device 400 and fall detection manager 422.

In addition, programming interfaces to the data stored as part of the computing device 400 and fall detection manager 422, can be available by standard mechanisms such as through C, C++, C#, and Java APIs; libraries for accessing files, databases, or other data repositories; scripting languages such as XML; or Web servers, FTP servers, NFS file servers, or other types of servers providing access to stored data. The fall detection data storage 416 and data repository 420 may be implemented as one or more database systems, file systems, or any other technique for storing such information, or any combination of the above, including implementations using distributed computing techniques.

Different configurations and locations of programs and data are contemplated for use with techniques described herein. A variety of distributed computing techniques are appropriate for implementing the components of the illustrated embodiments in a distributed manner including but not limited to TCP/IP sockets, RPC, RMI, HTTP, and Web Services (XML-RPC, JAX-RPC, SOAP, and the like). Other variations are possible. Other functionality could also be provided by each component/module, or existing functionality could be distributed amongst the components/modules in different ways, yet still achieve the functions of the fall detection manager 422.

Furthermore, in some embodiments, some or all of the components of the computing device 400 and fall detection manager 422 may be implemented or provided in other manners, such as at least partially in firmware and/or hardware, including, but not limited to one or more application-specific integrated circuits (“ASICs”), standard integrated circuits, controllers (e.g., by executing appropriate instructions, and including microcontrollers and/or embedded controllers), field-programmable gate arrays (“FPGAs”), complex programmable logic devices (“CPLDs”), and the like. Some or all of the system components and/or data structures may also be stored as contents (e.g., as executable or other machine-readable software instructions or structured data) on a computer-readable medium (e.g., as a hard disk; a memory; a computer network, cellular wireless network or other data transmission medium; or a portable media article to be read by an appropriate drive or via an appropriate connection, such as a DVD or flash memory device) so as to enable or configure the computer-readable medium and/or one or more associated computing systems or devices to execute or otherwise use, or provide the contents to perform, at least some of the described techniques.

In some embodiments, a computer-implemented method for fall detection includes: loading a decision tree on at least one sensor including an accelerometer, wherein the decision tree is trained based, at least in part, on automatic feature and filter selection and a size constraint for fitting into the at least one sensor, to detect falls of human beings; loading a finite state machine on the at least one sensor, wherein the finite state machine is configured, at least in part, to identify a set of phases of a fall independent from the decision tree; inputting data derived from at least the accelerometer into the decision tree and the finite machine; and combining output from the decision tree and output from the finite state machine via the at least one sensor to determine whether a fall has occurred.

In some embodiments, the output from the decision tree and the output from the finite state machine are combined using another finite state machine. In some embodiments, the other finite state machine resides within the at least one sensor.

In some embodiments, the output from the finite state machine is based, at least in part, on the output of the decision tree.

In some embodiments, the output from the finite state machine reduces false positive rate for the output from the decision tree.

In some embodiments, the combining comprises determining whether the finite state machine identifies the set of phases of a fall within a threshold of time of a fall being detected by the decision tree.

In some embodiments, a non-transitory computer-readable medium stores contents that cause one or more processors to perform actions. The actions include: implementing a decision tree that is trained based, at least in part, on automatic filter selection over first motion data, to detect falls of human beings; implementing a finite state machine configured, at least in part, to identify a set of phases of a fall independent from the decision tree; inputting second motion data into the decision tree and the finite machine; and combining output from the decision tree and output from the finite state machine to determine whether a fall has occurred.

In some embodiments, the second motion data includes accelerometer readings.

In some embodiments, the implementing of the decision tree, implementing of the finite state machine, and the combining of the outputs are performed on a sensor device. In some embodiments, the sensor device is attached to a person and capable of generating the second motion data.

In some embodiments, the output from the decision tree and the output from the finite state machine are combined using another finite state machine.

In some embodiments, wherein the output from the finite state machine is based, at least in part, on the output of the decision tree.

In some embodiments, the output from the finite state machine reduces false positive rate for the output from the decision tree.

In some embodiments, the combining comprises determining whether the finite state machine identifies the set of phases of a fall within a threshold of time of a fall being detected by the decision tree.

In some embodiments, a system includes one or more processors and a computing device coupled to the one or more processors and configured to perform actions.

The actions include: implementing a decision tree that is trained based, at least in part, on automatic filter selection over first motion data, to detect falls of human beings; implementing a finite state machine configured, at least in part, to identify a set of phases of a fall independent from the decision tree; inputting second motion data into the decision tree and the finite machine; and combining output from the decision tree and output from the finite state machine to determine whether a fall has occurred.

In some embodiments, the output from the decision tree and the output from the finite state machine are combined using another finite state machine.

In some embodiments, the output from the finite state machine reduces false positive rate for the output from the decision tree.

In some embodiments, the combining comprises determining whether the finite state machine identifies the set of phases of a fall within a threshold of time of a fall being detected by the decision tree.

In some embodiments, the first motion data includes accelerometer readings.

In some embodiments, the computing device is attachable to a person and capable of generating accelerometer readings.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A computer-implemented method for fall detection, the method comprising:
loading a decision tree on at least one sensor including at least one of an accelerometer, gyroscope, or magnetometer, wherein the decision tree is trained based, at least in part, on automatic feature and filter selection and a size constraint for fitting into the at least one sensor, to detect falls of human beings;
loading a finite state machine on the at least one sensor, wherein the finite state machine is configured, at least in part, to identify a set of phases of a fall independent from the decision tree;
inputting data derived from at least the accelerometer into the decision tree and the finite machine; and
combining output from the decision tree and output from the finite state machine via the at least one sensor to determine whether a fall has occurred.

2. The method of claim 1, wherein the output from the decision tree and the output from the finite state machine are combined using another finite state machine.

3. The method of claim 2, wherein the other finite state machine resides within the at least one sensor.

4. The method of claim 1, wherein the output from the finite state machine is based, at least in part, on the output of the decision tree.

5. The method of claim 1, wherein the output from the finite state machine reduces false positive rate for the output from the decision tree.

6. The method of claim 1, wherein the combining comprises determining whether the finite state machine identifies the set of phases of a fall within a threshold of time of a fall being detected by the decision tree.

7. A non-transitory computer-readable medium storing contents that cause one or more processors to perform actions comprising:
implementing a decision tree that is trained based, at least in part, on automatic filter and feature selection over first motion data, to detect falls of human beings;
implementing a finite state machine configured, at least in part, to identify a set of phases of a fall independent from the decision tree;
inputting second motion data into the decision tree and the finite machine; and
combining output from the decision tree and output from the finite state machine to determine whether a fall has occurred.

8. The non-transitory computer-readable medium of claim 7, wherein the second motion data includes accelerometer readings.

9. The non-transitory computer-readable medium of claim 7, wherein the implementing of the decision tree, implementing of the finite state machine, and the combining of the outputs are performed on a sensor device.

10. The non-transitory computer-readable medium of claim 9, wherein the sensor device is attached to a person and capable of generating the second motion data.

11. The non-transitory computer-readable medium of claim 7, wherein the output from the decision tree and the output from the finite state machine are combined using another finite state machine.

12. The non-transitory computer-readable medium of claim 7, wherein the output from the finite state machine is based, at least in part, on the output of the decision tree.

13. The non-transitory computer-readable medium of claim 7, wherein the output from the finite state machine reduces false positive rate for the output from the decision tree.

14. The non-transitory computer-readable medium of claim 7, wherein the combining comprises determining whether the finite state machine identifies the set of phases of a fall within a threshold of time of a fall being detected by the decision tree.

15. A system, comprising:
one or more processors; and
a computing device coupled to the one or more processors and configured to perform actions comprising:
implementing a decision tree that is trained based, at least in part, on automatic filter and feature selection over first motion data, to detect falls of human beings;
implementing a finite state machine configured, at least in part, to identify a set of phases of a fall independent from the decision tree;
inputting second motion data into the decision tree and the finite machine; and
combining output from the decision tree and output from the finite state machine to determine whether a fall has occurred.

16. The system of claim 15, wherein the output from the decision tree and the output from the finite state machine are combined using another finite state machine.

17. The system of claim 15, wherein the output from the finite state machine reduces false positive rate for the output from the decision tree.

18. The system of claim 15, wherein the combining comprises determining whether the finite state machine identifies the set of phases of a fall within a threshold of time of a fall being detected by the decision tree.

19. The system of claim 15, wherein the first motion data includes accelerometer readings.

20. The system of claim 15, wherein the computing device is attachable to a person and capable of generating accelerometer readings.
